(19) United States
(12) Patent Application Publication
(10) Pub. No.: US 2019/0062704 A1
(43) Pub. Date: Feb. 28, 2019

(54) METHOD OF CELL CULTURE

(71) Applicant: 3D Global Biotech Inc., New Taipei City (TW)

(72) Inventor: KENG-LIANG OU, New Tapei City (TW)

(21) Appl. No.: 15/967,401

(22) Filed: Apr. 30, 2018

(30) Foreign Application Priority Data
Aug. 31, 2017 (TW) 106129748

(51) Int. Cl. C12N 5/0797; C12N 5/00

(52) U.S. Cl. CPC C12N 5/0623; C12N 5/0068; C12N 2500/25; C12N 2539/00; C12N 2501/375; C12N 2502/085; C12N 2533/30; C12N 2500/32

(57) ABSTRACT

The present invention discloses a method for culturing human limbal stem cells comprising the steps of: pre-treating a feeder cell; seeding the feeder cells in a porous membrane; placing the porous membrane in a container to separate the container into a first cell culture compartment and a second cell culture compartment, wherein the feeder cells are located in the second cell culture compartment; injecting the culture medium into the container; and placing the human limbal stem cells in the first cell culture compartment. Compared to the prior art, the method for culturing human limbal stem cells of the present invention utilizes the porous membrane to culture the human limbal stem cells to make the expansion rate better than the traditional culture method. In addition, the culture medium used in the present invention does not contain the dimethyl sulfoxide (DMSO) with cytotoxicity to reduce the probability of cell death.

### CROSS-REFERENCE TO RELATED APPLICATIONS

This application claims priority to Taiwan application number 106129748 filed Aug. 31, 2017, reference of which is hereby incorporated in its entirety.

### Field of the Invention

The present invention relates to a method of cell culture, more particularly, to a method of cell culture using porous membranes.

### Description of the Prior

Normal and stable corneal surface is essential for maintaining corneal transparency and maintaining normal physiological function. Corneal defects are mainly caused by trauma, bacterial infection, chemical burns and contact lenses. According to statistics, about 13 million people around the world suffer from corneal injury or disease affecting vision, and 1500 to 2 million new cases are diagnosed each year. Corneal defects may lead to a series of eye diseases even the blindness.

Limbal stem cells (LSCs) are not only the embankment between the cornea and conjunctiva, but also the source of corneal epithelial regeneration. The discovery of the limbal stem cells is one of the most important advances in ophthalmology in recent decades. For the time being, limbal stem cell transplantation is the most effective treatment for curing the surface disease and reconstructing the corneal structure. The standard method of culturing limbal stem cells in vitro is to co-culture limbal stem cells directly on the feeder cells arrest of growth. During the culture, feeder cells secrete cytokines to assist the proliferation of limbal stem cells. However, the limbal stem cells colony is formed with the cell proliferation to push the feeder cells aside, so the limbal stem cells may be insufficiently supplied by feeder cells. Besides, since the limbal stem cells are contacted to the feeder cells directly in culture, the feeder cells are difficult to be removed clearly while collecting the limbal stem cells. As a result, the risk of cross-contamination is generated.

### SUMMARY OF THE INVENTION

In view of this, the present invention provides a method of cell culture comprising the following steps: treating a feeder cell with a mitomycin C in constant temperature, wherein the concentration range of the mitomycin C is between 1 to 25 μg/mL, and the treating time is between 2 hours to 2 hours and 45 minutes, and then removing the mitomycin C; mixing the treated feeder cell with a minor medium, and seeding the feeder cell on a porous membrane; depositing the porous membrane seeded with the feeder cell into a container, so that the container is separated to a first culture compartment and a second culture compartment, wherein the feeder cell is located in the second culture compartment; pouring a major medium into the container to fill the first culture compartment and the second culture compartment; and depositing a human limbal stem cell into the first culture compartment.

In an embodiment, the concentration of the mitomycin C is 5 μg/mL, and the treating time of the feeder cell with the mitomycin C is 2 hours and 15 minutes.

In an embodiment, the porous membrane is made of polyethylene terephthalate (PET) and the pore diameter of the porous membrane is 0.4 μm. The porous membrane allows cytokine to be released by the feeder cells to pass through to feed the human limbal stem cell for amplifying the human limbal stem cell while the feeder cell and the human limbal stem cell are prevented from contacting directly by the porous membrane.

In an embodiment, the major medium is a medium with the same components as the minor medium. The medium is based on Dulbecco's Minimum Essential Medium/F12 (DMEM/F12). The component of the medium comprises L-glutamine, triiodothyronine, insulin, transferrin, and selenous acid, and the dimethyl sulfoxide (DMSO) content is less than 0.1%.

In practice, the feeder cell is Swiss-3T3 fibroblast.

Compare to prior art, the method of cell culture of the present invention uses a unique culture medium and culture parameters with the culture technique of porous membrane to culture human limbal stem cells, so that the expansion rate is better than the traditional culture method. Besides, the culture medium used in the present invention does not contain dimethyl sulfoxide with cytotoxicity, so that the probability of cell death is reduced.

### BRIEF DESCRIPTION OF THE APPENDED DRAWINGS

Some of the embodiments will be described in detail, with reference to the following figures, wherein like designations denote like members, wherein:

FIG. 1 shows a flow chart diagram of an embodiment of the method of cell culture of the present invention.

FIG. 2 shows an advanced flow chart diagram of an embodiment of pre-treating the feeder cells.

FIG. 3 shows an advanced flow chart diagram of an embodiment of the method of cell culture of the present invention.

FIG. 4 shows a schematic diagram of an embodiment of pre-treating the feeder cells.

FIG. 5 shows a schematic diagram of an embodiment of seeding the feeder cells.

FIG. 6 shows a schematic diagram of an embodiment of the step of depositing the porous membrane into a container.

FIG. 7 shows a schematic diagram of an embodiment of the step of pouring medium.

FIG. 8 shows a schematic diagram of an embodiment of the step of depositing human limbal stem cells.

FIG. 9 shows a flow chart diagram of an embodiment of the traditional method of cell culture.

FIG. 10 shows a schematic diagram of an embodiment of the traditional method of cell culture.

FIG. 11(A) shows a two-dimensional dot chart diagram of flow cytometry of the experiment one.

FIG. 11(B) shows a two-dimensional dot chart diagram of flow cytometry of the experiment two.

FIG. 12 shows the expansion rate of the experiment one and experiment two.

FIG. 13(A) shows a cell colony image of the experiment one.

FIG. 13(B) shows a cell colony image of the experiment two.

FIG. 14 shows an analysis bar chart of colony formation of the experiment one and experiment two.

FIG. 15 shows an analysis bar chart of colony size of the experiment one and experiment two.

The advantages, spirits, and features of the present invention will be explained and discussed with embodiments and figures as follows.

### DETAILED DESCRIPTION OF THE INVENTION

A detailed description of the hereinafter described embodiments of the disclosed apparatus and method are presented herein by way of exemplification and not limitation with reference to the Figures. Although certain embodiments are shown and described in detail, it should be understood that various changes and modifications can be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

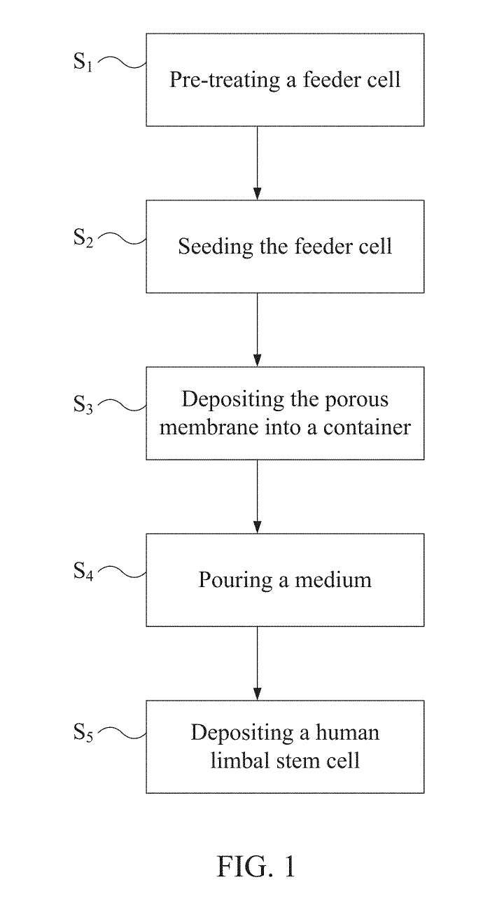

Please refer to FIG. 1. FIG. 1 shows a flow chart diagram of an embodiment of the method of cell culture of the present invention. The present invention provides a method of cell culture comprising the following steps: S1: Pre-treating a feeder cell; S2: Seeding the feeder cell on a porous membrane; S3: Depositing the porous membrane into a container; S4: Pouring a medium into the container; and S5: Depositing a human limbal stem cell into the container to culture the cell.

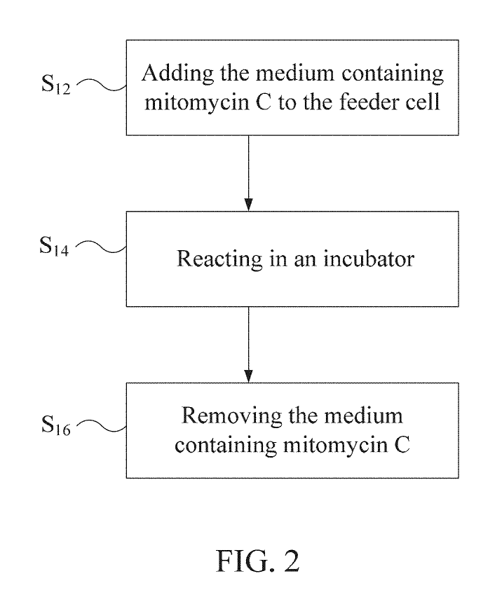

Please refer to FIG. 2. FIG. 2 shows an advanced flow chart diagram of an embodiment of pre-treating the feeder cells 100. In an embodiment of the present invention, the step of S1: pre-treating a feeder cell further comprises the following steps: S12: Adding the medium containing mitomycin C to the feeder cells; S14: Reacting in an incubator; and S16: Removing the medium containing mitomycin C.

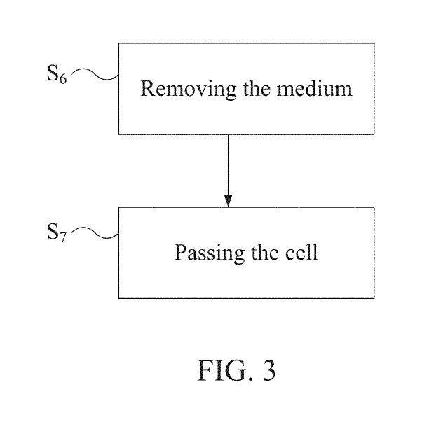

Please refer to FIG. 3. FIG. 3 shows an advanced flow chart diagram of an embodiment of the method of cell culture of the present invention. After the step S5 about 3 to 5 days, execute the step S6: Removing the medium; and then the step S7: Passing the cells.

The operation process of steps S1 to S5 will be described schematically below.

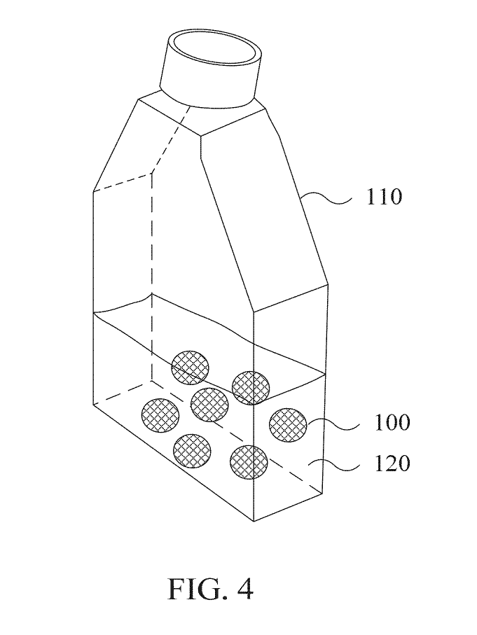

Please refer to FIG. 1, FIG. 2, and FIG. 4. FIG. 4 shows a schematic diagram of an embodiment of pre-treating the feeder cells 100. In an embodiment, the Swiss-3T3 fibroblast cell line is used as the feeder cell 100. The feeder cells 100 are placed into a cell culture flask 110 such as T-75 flask. A fibroblast growth medium 120 added with the mitomycin C with concentration 1 μg/mL to 25 μg/mL is poured into the cell culture flask 110. Wherein, the better adding concentration of mitomycin C is 5 μg/mL. Next, the cell culture flask 110 is placed into an incubator at 37° C. for 2 hours to 2 hours and 45 minutes. Wherein, the better reaction time is 2 hours and 15 minutes. After the reaction, the fibroblast growth medium 120 added with the mitomycin C is withdrawn from the cell culture flask 110. The feeder cells 100 are washed with 1× PBS (phosphate buffered saline). The feeder cells 100 after washing are centrifuged at 300 g for 3 minutes in the centrifuge to complete the process of pre-treating the feeder cells 100. The purpose of the pre-treating step S1 is to inhibit the cell division and amplification while maintaining the cell physiological metabolism. Therefore, the feeder cells 100 can provide the cytokine to the human limbal stem cells normally without competing for the nutrition of the medium in the co-culture process of the feeder cells 100 and the human limbal stem cells.

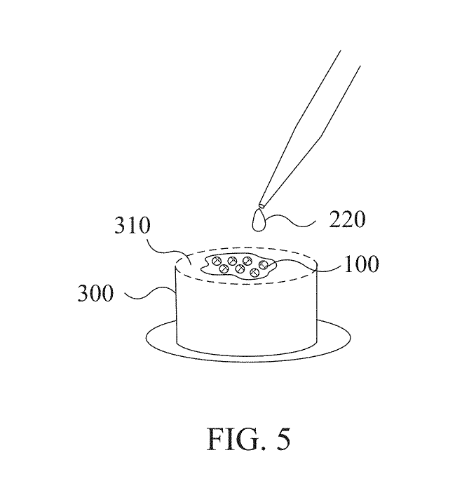

Please refer to FIG. 1 and FIG. 5. FIG. 5 shows a schematic diagram of an embodiment of seeding the feeder cells 100. In an embodiment, the feeder cells 100 after pre-treating are mixed with a limbal stem cell culture medium 220 (minor medium). Next, the limbal stem cell culture medium 220 containing the feeder cells 100 is added to the bottom 310 of the porous membrane 300. Wherein, the better seeding density is 2×10⁴ cells/cm². After a period of time (about 2.5 to 4 hours), the step S2 is finished while the feeder cell 100 is seeding at the bottom 310 of the porous membrane 300 completely.

As shown in FIG. 5, the shape of the porous membrane 300 is similar to a hat with a brim. The porous membrane 300 consists of essentially of polyethylene terephthalate (PET). The bottom 310 of the porous membrane 300 has pores distributed evenly. The porous membrane 300 used in the present invention has a pore size of 0.4 μm. This pore size allows cytokines to pass through while blocking cells penetration.

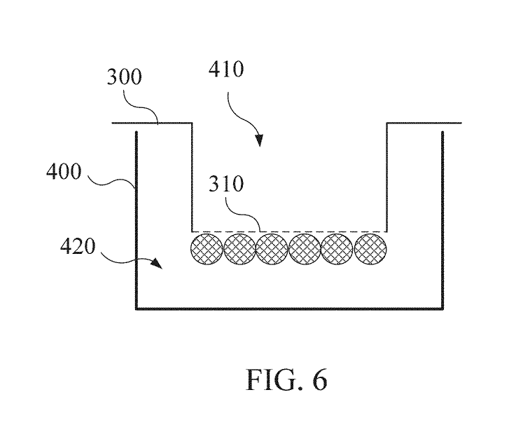

Please refer to FIG. 1 and FIG. 6. FIG. 6 shows a schematic diagram of an embodiment of the step S3 of depositing the porous membrane 300 into a container 400. In an embodiment, the porous membrane 300 seeded with the feeder cells 100 is inverted and placed into the container 400, so that the edge of the porous membrane 300 is engaged with the edge of the container 400. Wherein, the container 400 is a well of the 6-well plates. As shown in FIG. 6, the container 400 is separated into a first cell culture compartment 410 and a second cell culture compartment 420. Wherein, the feeder cells are located in the second cell culture compartment 420 and attached to the bottom 310 of the porous membrane 300.

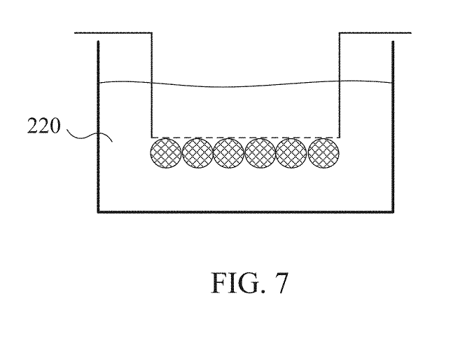

Please refer to FIG. 1 and FIG. 7. FIG. 7 shows a schematic diagram of an embodiment of the step S4 of pouring medium. In an embodiment, the limbal stem cell culture medium 220 (major medium) is pouring into the container 400 to fill the first culture compartment 410 and the second culture compartment 420. It should be noted that the major medium used in this step S4 is the same as the secondary medium used in step S2 as the limbal stem cell culture medium 220. Therefore, the medium is not necessary to be removed after seeding the feeder cells 100 of the step S2, so that the nutrition can be conserved completely.

In practice, the limbal stem cell culture medium 220 is based on Dulbecco's Minimum Essential Medium/F12 (DMEM/F12). The added components of the limbal stem cell culture medium 220 contain human epidermal growth factor 20 ng/mL, hydrocortisone 0.5 μg/mL, fetal bovine serum (FBS) 5%, L-glutamine 4 mM, triiodothyronine 2 nM, insulin 5 μg/mL, transferrin 5 μg/mL, selenous acid 5 ng/mL, penicillin 100 μg/mL, streptomycin 100 U/mL, amphotericin B 1.25 μg/mL, gentamycin 50 μg/mL. It should be noted that dimethyl sulfoxide (DMSO) often used in the known medium is less than 0.1% in the culture medium of the present invention. A small amount of dimethyl sulfoxide can promote cell differentiation; also, dimethyl sulfoxide may inhibit cells proliferation due to the cytotoxicity. Excessive dimethyl sulfoxide may even cause the cell death through the mechanism such as apoptosis or necrosis. Therefore, the limbal stem cell culture medium 220 does not contain dimethyl sulfoxide to prevent the cells from death.

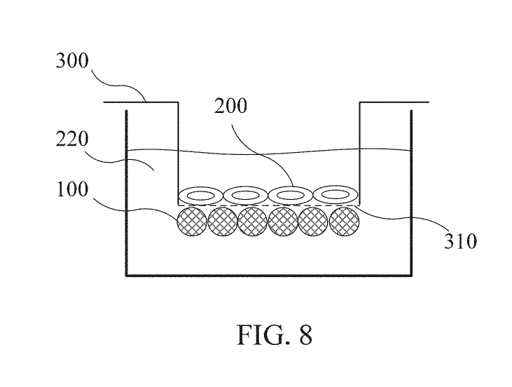

Please refer to FIG. 1 and FIG. 8. FIG. 8 shows a schematic diagram of an embodiment of the step S5 of depositing human limbal stem cells. In an embodiment, the human limbal stem cells (LSCs) 200 are disposed into the first culture compartment 410 filled with the limbal stem cell culture medium 220. The feeder cells 100 secrete the cytokines in the limbal stem cell culture medium 220. The cytokines are transferred to the human limbal stem cells 200 through the bottom 310 of the porous membrane 300. At the meanwhile, the porous membrane 300 separates the feeder cells 100 and the human limbal stem cells 200 to avoid the contamination of direct contact.

Please refer to FIG. 3 again. The feeder cells 100 decease gradually after co-culturing of the step S5 about 3 to 5 days. The nutrition of the limbal stem cell culture medium 220 decreases and the human limbal stem cells 200 are amplified significantly. Next, the step S6 is executed. The limbal stem cell culture medium 220 is removed with the feeder cells 100. The human limbal stem cells 200 are washed with 1× PBS. After that, the trypsin-EDTA is added to the human limbal stem cells 200 for 8 minutes in the incubator, so that the amplified human limbal stem cells 200 are detached to subculture. Wherein, the process of the subculture is to re-separate the amplified human limbal stem cells 200 to the new container 400. The new container 400 is poured with the new limbal stem cell culture medium 220 and the pre-treating feeder cells 100 (repeat the steps of S1-S5), so that the human limbal stem cells 200 are amplified continuously.

The amplification of the cultured human limbal stem cells 200 following the cultured method and the parameters of the present invention is better than the traditional culture method, and the method, parameters, result, and the effect will be illustrated by the following experiment.

In experiment one, the human limbal stem cells 200 are cultured with the cultured method of the present invention. The experiment method and the better parameter of the step S1-S7 in the mentioned embodiment are used to culture the human limbal stem cells 200.

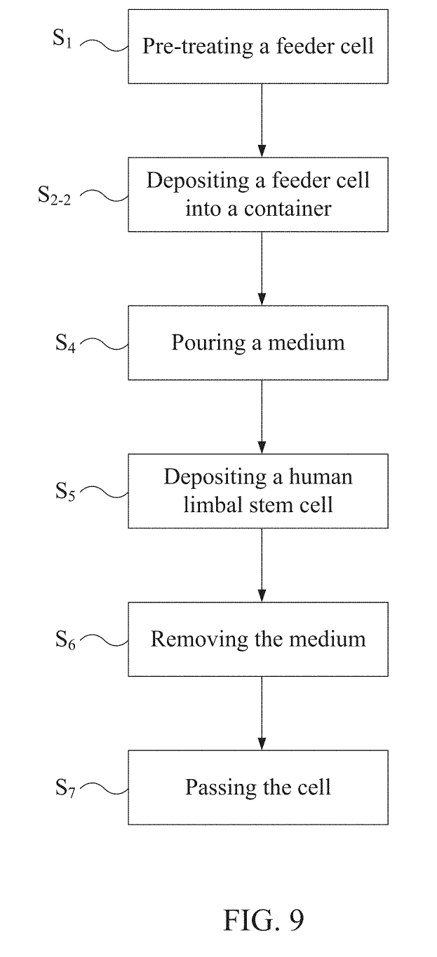

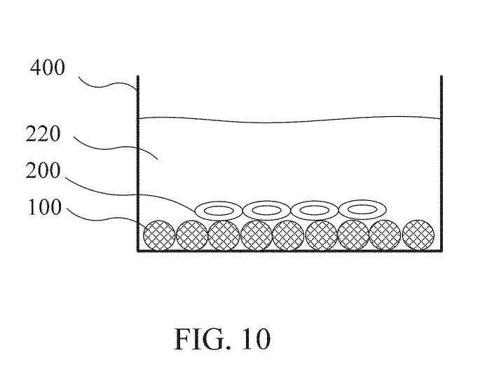

In experiment two, the human limbal stem cells 200 are cultured with the traditional method. Please refer to FIG. 9 and FIG. 10. FIG. 9 shows a flow chart diagram of an embodiment of the traditional method of cell culture 200. FIG. 10 shows a schematic diagram of an embodiment of the traditional method of cell culture 200. In an embodiment, the traditional method of cell culture 200 is considered as the control group, comprising S1: Pre-treating a feeder cells 100; S2-2: Seeding the feeder cells 100 into a container 400; S4: Pouring the limbal stem cell culture medium 220 into the container 400; S5: Depositing the human limbal stem cells 200 into the container 400; After the step S5 about 3 to 5 days, S6: Removing the limbal stem cell culture medium 220 and the feeder cells 100; and the S7: Passing the cells, and re-separating the human limbal stem cells 200 to the new container 400 and adding the new limbal stem cell culture medium 220 and the feeder cells 100. The schematic diagram of the traditional method of cell culture 200 is shown in FIG. 10. In contrast to the cultured method of the present invention, the traditional method deposits the human limbal stem cells 200 on the feeder cells 100 directly. This cultured method may result in increasing the probability of contaminating the human limbal stem cells 200, also, the human limbal stem cells 200 and the feeder cells 100 are difficult to separate. Wherein, the component of the medium and the parameters of pre-treating in this embodiment are the same as the experiment one.

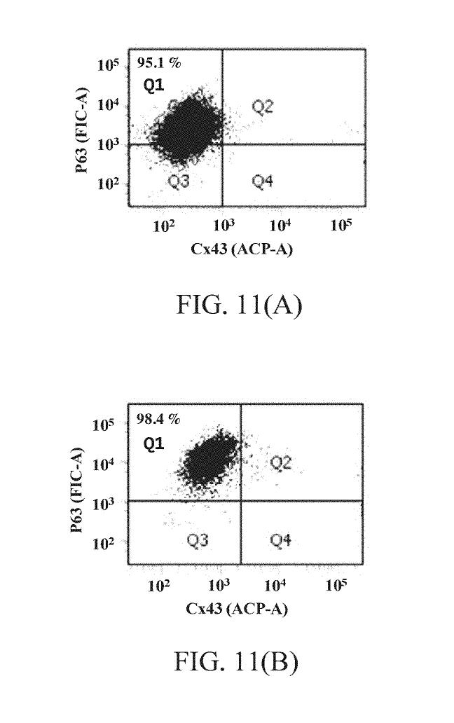

Result 1: Compare the ratio of stem cells of experiment one to experiment two. Please refer to FIG. 11(A) and FIG. 11(B). FIG. 11(A) shows a two-dimensional dot chart diagram of flow cytometry of the experiment one. FIG. 11(B) shows a two-dimensional dot chart diagram of flow cytometry of the experiment two. In this result, the flow cytometry is used to detect the stem cells ratio after culturing the human limbal stem cells 200 for 7 days in experiment one and experiment two. Wherein, the detected surface marker P63 protein expression is considered as the positive indicator, and the protein expression of C×43 is considered as the negative indicator. That is to say, if the higher the protein P63 expression or the lower the protein C×43, the more obvious the characteristics of stem cells. The human limbal stem cells 200 are detected by double staining flow cytometry assay, and the X axial in the FIG. 11(A) and FIG. 11(B) is a fluorescent dye APC-A for observing the expressed amount of protein C×43, and the Y axial is a fluorescent dye FITC-A for observing the expressed amount of protein P63. Each point in the FIG. 11(A) and FIG. 11(B) represent a cell. The cell distributing position closer to the Q1 quadrant in figure represents the characteristics of stem cell more obviously. As can be realized from FIG. 11(A) and FIG. 11(B), the ratio of cells in Q1 quadrant in experiment one is 95.1% and the ratio of cells in Q1 quadrant in experiment two is 98.4% cells. There is no significant difference in the ratio of cells in Q1 quadrant between the two experiments.

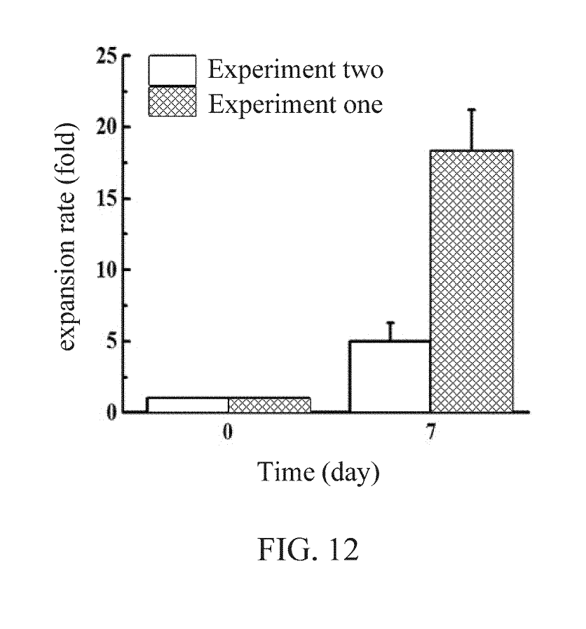

Result 2: Compare the expansion rate of stem cells of experiment one to experiment two. Please refer to FIG. 12. FIG. 12 shows the expansion rate of the experiment one and experiment two. The expansion rate is defined as the number of cells after culture divided by the original number of cells. FIG. 12 is the expansion rate chart bar diagram of culturing the human limbal stem cells 200 for 7 days in experiment one and experiment two. As shown in FIG. 12, the expansion rate in experiment two is 4.99 fold, and the expansion rate in experiment one is 18.3 fold. Therefore, the expansion rate of the present invention method is significantly better than the traditional cultured method.

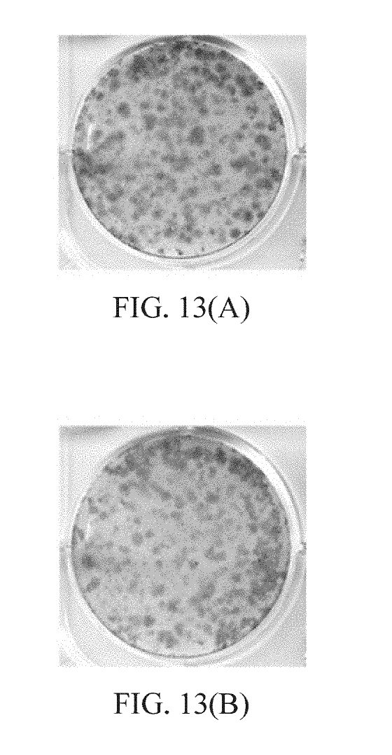

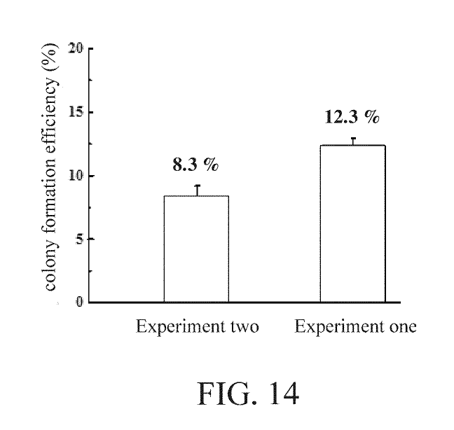

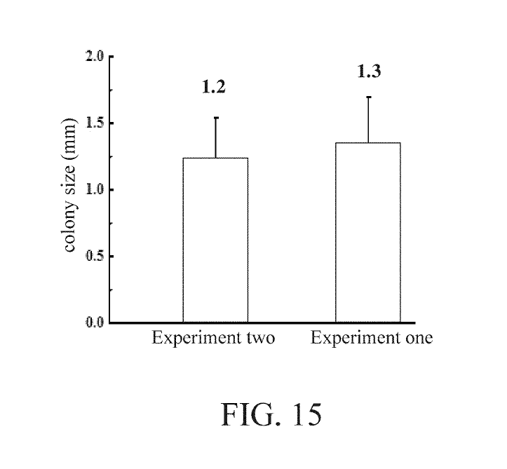

Result 3: Compare the cell colony property of stem cells of experiment one to experiment two. Please refer to FIG. 13(A), FIG. 13(B), FIG. 14, and FIG. 15. FIG. 13(A) shows a cell colony image of the experiment one. FIG. 13(B) shows a cell colony image of the experiment two. FIG. 14 shows an analysis bar chart of colony formation of the experiment one and experiment two. FIG. 15 shows an analysis bar chart of colony size of the experiment one and experiment two. Cell colony formation reflects the ability of cell division. The more colonies are formed, the more precursor cells have the ability to divide. Since the stem cells have the property of forming colony, the colony analysis assay can be an indicator to estimate the property of the stem cells. As shown in FIG. 13(A) and FIG. 13(B), the property of cell forming colony in experiment one is more obviously than experiment two. The experimental results of FIG. 13(A) and FIG. 13(B) are performed image analysis by microscope to obtain the quantitative data, and the data are represented by bar chart as shown in FIG. 14, and FIG. 15. In FIG. 14, colony formation efficiency in experiment one is 12.3%, higher than 8.3% in experiment two. Further, the sizes of the formation colonies are compared. In FIG. 15, the cell colony size of experiment one is 1.3 mm, also higher than 1.2 mm in experiment two.

Compare to prior art, the method of cell culture of the present invention uses a unique culture medium and culture parameters with the culture technique of porous membrane to culture human limbal stem cells. It can be known from the mentioned experiment and result, both the expansion rate and the properties of the cell colony in the present invention are obviously better than the traditional culture method. Besides, the culture medium used in the present invention does not contain dimethyl sulfoxide with cytotoxicity, so that the probability of cell death is reduced.

With the examples and explanations mentioned above, the features and spirits of the invention are hopefully well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A method of cell culture, comprising the following steps:
treating a feeder cell with a mitomycin C in constant temperature, wherein the concentration range of the mitomycin C is between 1 to 25 μg/mL, and the treating time is between 2 hours to 2 hours and 45 minutes, and then removing the mitomycin C;
mixing the treated feeder cell with a minor medium, and seeding the feeder cell on a porous membrane;
depositing the porous membrane seeded with the feeder cell into a container, so that the container is separated to a first culture compartment and a second culture compartment, wherein the feeder cell is located in the second culture compartment;
pouring a major medium into the container to fill the first culture compartment and the second culture compartment; and
depositing a human limbal stem cell into the first culture compartment.

2. The method of cell culture of claim 1, wherein the concentration of the mitomycin C is 5 μg/mL, and the treating time of the feeder cell with the mitomycin C is 2 hours and 15 minutes.

3. The method of cell culture of claim 1, wherein the feeder cell releases a cytokine in the major medium; the porous membrane allows the cytokine to pass through the first culture compartment and the second culture compartment, but the feeder cell and the human limbal stem cell are prevented from passing through by the porous membrane.

4. The method of cell culture of claim 1, wherein the porous membrane is made of polyethylene terephthalate (PET) and the pore diameter of the porous membrane is 0.4 μm.

5. The method of cell culture of claim 1, wherein the major medium is a medium with the same components as the minor medium.

6. The method of cell culture of claim 5, wherein the medium comprises L-glutamine, triiodothyronine, insulin, transferrin, and selenous acid, and the dimethyl sulfoxide (DMSO) content is less than 0.1%.

7. The method of cell culture of claim 6, wherein the concentration of L-glutamine in the medium is 4 mM, and the concentration of triiodothyronine in the medium is 2 nM, and the concentration of insulin in the medium is 5 μg/mL, and the concentration of transferrin in the medium is 5 μg/mL, and the concentration of selenous acid in the medium is 5 ng/mL.

8. The method of cell culture of claim 7, wherein the medium comprises Dulbecco's Minimum Essential Medium/F12 (DMEM/F12).

9. The method of cell culture of claim 1, wherein the feeder cells are seeded on the porous membrane at the density of 2×10⁴ cells/cm².

10. The method of cell culture of claim 1, wherein the feeder cell is Swiss-3T3 fibroblast.
